(19) United States
(12) Patent Application Publication
(10) Pub. No.: US 2021/0085191 A1
(43) Pub. Date: Mar. 25, 2021

(54) ELECTRONIC DEVICE AND METHOD OF ESTIMATING PHYSIOLOGICAL SIGNAL

(71) Applicant: Samsung Electronics Co., Ltd., Suwon-si (KR)

(72) Inventors: Illya DEGTYARENKO, Kyiv (UA); Kostyantyn SLYUSARENKO, Kyiv (UA); Jooman HAN, Suwon-si (KR); Jinwoo SEO, Suwon-si (KR); Jongin PARK, Suwon-si (KR)

(21) Appl. No.: 16/983,627

(22) Filed: Aug. 3, 2020

(30) Foreign Application Priority Data
Sep. 20, 2019 (KR) 10-2019-0116027

(51) Int. Cl. A61B 5/0205; A61B 5/11; A61B 5/16; A61B 5/00; A61B 5/05

(52) U.S. Cl. CPC A61B 5/0205; A61B 5/1123; A61B 5/165; A61B 5/0077; A61B 5/021; A61B 5/1118; A61B 5/7264; A61B 5/742; A61B 5/05

(57) ABSTRACT

An electronic device is provided. The electronic device includes a first sensor configured to measure a physiological parameter of a user, a second sensor configured to measure motion feature of the user, at least one processor operatively connected with the first sensor or the second sensor, and a memory operatively connected with the at least one processor, wherein the memory stores instructions executed to enable the at least one processor to, monitor a physiological signal of the user based on the physiological parameter measured by the first sensor, upon detecting motion of the user using at least one of the first sensor or the second sensor while monitoring the physiological signal, identify a first signal indicating a physiological activity trend based on the motion feature obtained from the second sensor, and correct the physiological signal recorded for a first time period during which the motion is maintained based on the first signal.

### CROSS-REFERENCE TO RELATED APPLICATION(S)

This application is based on and claims priority under 35 U.S.C. § 119(a) of a Korean patent application number 10-2019-0116027, filed on Sep. 20, 2019, in the Korean Intellectual Property Office, the disclosure of which is incorporated by reference herein in its entirety.

### BACKGROUND

### 1. Field

The disclosure relates to a concern electronic devices and methods of monitoring physiological biometric signals stably regardless of the user's posture or motion.

### 2. Description of Related Art

As information technology develops, various types of wearable devices, such as watches, glasses, shoes, or clothing, come on the market, with wearable devices becoming a must-have item along with smartphones. Wearable devices are able to gather data in real-time by interworking with a smartphone. With the ease to carry and use, wearable devices are spreading into medical, healthcare, and other various industry sectors.

The recent growing interest in well-being is leading to vigorous research efforts at real-time monitoring of users' health condition using a sensor-equipped wearable device. For example, a photoplethysmography (PPG) sensor may be included in a wearable device to real-time monitor health information, such as the heart rate or heart rate variability of the user wearing the wearable device. The results of measurement may be continuously analyzed to determine the user's health condition while preventing physical activity that may overburden the user's body.

A PPG sensor equipped in a wearable device converts the photoplethysmogram per body portion into a PPG signal, thereby measuring the user's heart rate. The PPG signal indicates a variation in the volume of blood vessels using the absorbance of light radiated from a light emitting diode (LED) to and reflected by the skin. Real-time estimation of the user's heart rate may be rendered possible by detecting a variation in the volume of blood vessels that occurs when the heart contracts and relaxes, by the PPG signal. However, the PPG sensor is vulnerable to motion artifacts due to the user's motion and may easily be distorted by the user's posture or motion. The PPG signal measured while the user wearing the wearable device moves may have a motion artifact component, which may deteriorate the accuracy of measurement and reliability of health condition analysis.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide more accurate and reliable physiological biometric information using a wearable device regardless of the user's posture or motion by estimating physiological signals considering the physiological activity trend according to the motion feature upon detecting the user's motion.

Additional aspects will be set forth in part in the description which follows and, in part, will be apparent from the description, or may be learned by practice of the presented embodiments.

In accordance with an aspect of the disclosure, an electronic device is provided. The electronic device includes a first sensor configured to measure a physiological parameter for a user, a second sensor configured to measure the user's motion feature, at least one processor operatively connected with the first sensor or the second sensor, and a memory operatively connected with the at least one processor, wherein the memory stores instructions executed to enable the at least one processor to, monitor the user's physiological signal based on the physiological parameter measured by the first sensor, upon detecting the user's motion using at least one of the first sensor or the second sensor while monitoring the user's physiological signal, identify a first signal indicating a physiological activity trend based on the user's motion feature obtained from the second sensor, and correct the user's physiological signal recorded for a first time period during which the user's motion is maintained based on the first signal.

In accordance with another aspect of the disclosure, a method of estimating a physiological signal by an electronic device is provided. The method includes a first sensor configured to measure a user's physiological parameter and a second sensor configured to measure the user's motion feature comprises monitoring the user's physiological signal based on the physiological parameter measured by the first sensor, detecting the user's motion using at least one of the first sensor or the second sensor while monitoring the user's physiological signal, identifying a first signal indicating a physiological activity trend based on the user's motion feature obtained from the second sensor in response to detecting the user's motion, and correcting the user's physiological signal recorded for a first time period during which the user's motion is maintained based on the first signal.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be apparent from the following detailed description taken in conjunction with the accompanying drawings, wherein:

FIGS. 1A and 1B illustrate measuring a physiological signal using a wearable device according to various embodiments of the disclosure;

FIGS. 2A, 2B, and 2C illustrate a physiological signal, signal quality and acceleration signal measured corresponding to whether the user moves according to an embodiment of the disclosure;

FIG. 3 is a block diagram 300 illustrating an electronic device according to an embodiment of the disclosure;

FIGS. 4A and 4B illustrate a method of correcting physiological signals when a user moves according to various embodiments of the disclosure;

FIG. 4C illustrates a method of estimating a physiological signal in a period during which a user moves according to an embodiment of the disclosure;

FIG. 5 illustrates a process of estimating a physiological signal in a period during which a user moves according to an embodiment of the disclosure;

FIG. 6 illustrates a process of estimating a physiological signal using a physiological activity prediction model in a period during which a user moves according to an embodiment of the disclosure;

FIGS. 7A, 7B, and 7C illustrate a physiological activity prediction model according to various embodiments of the disclosure;

FIG. 8 is a flowchart illustrating a method of estimating a physiological signal by an electronic device according to an embodiment of the disclosure; and

FIG. 9 illustrates providing physiological information estimated corresponding to a motion feature by a smartwatch according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

As used herein, the terms “have,” “may have,” “include,” or “may include” a feature (e.g., a number, function, operation, or a component, such as a part) indicate the existence of the feature and do not exclude the existence of other features.

As used herein, the terms “configured (or set) to” may be interchangeably used with the terms “suitable for,” “having the capacity to,” “designed to,” “adapted to,” “made to,” or “capable of” depending on circumstances. The term “configured (or set) to” does not essentially mean “specifically designed in hardware to.” Rather, the term “configured to” may mean that a device can perform an operation together with another device or parts. For example, the term “processor configured (or set) to perform A, B, and C” may mean a generic-purpose processor (e.g., a central processing unit (CPU) or application processor) that may perform the operations by executing one or more software programs stored in a memory device or a dedicated processor (e.g., an embedded processor) for performing the operations.

Each of the aforementioned components of the electronic device may include one or more parts, and a name of the part may vary with a type of the electronic device. The electronic device in accordance with various embodiments of the disclosure may include at least one of the aforementioned components, omit some of them, or include other additional component(s). Some of the components may be combined into an entity, but the entity may perform the same functions as the components may do.

The terms as used herein are provided merely to describe some embodiments thereof, but not to limit the scope of other embodiments of the disclosure. All terms including technical and scientific terms used herein have the same meaning as commonly understood by one of ordinary skill in the art to which the embodiments of the disclosure belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein. In some cases, the terms defined herein may be interpreted to exclude embodiments of the disclosure.

Examples of the electronic device may include at least one of a smartphone, a tablet personal computer (PC), a mobile phone, a video phone, an e-book reader, a desktop PC, a laptop computer, a netbook computer, a workstation, a PDA (personal digital assistant), a portable multimedia player (PMP), a motion pictures expert group (MPEG-1 or MPEG-2) audio layer 3 (MP3) player, a mobile medical device, a camera, or a wearable device (e.g., smart glasses, a head-mounted device (HMD), electronic clothes, an electronic bracelet, an electronic necklace, an electronic appcessory, an electronic tattoo, a smart mirror, or a smart watch).

According to an embodiment of the disclosure, the electronic device may be a smart home appliance. Examples of the smart home appliance may include at least one of a television, a digital video disk (DVD) player, an audio player, a refrigerator, an air conditioner, a cleaner, an oven, a microwave oven, a washer, a drier, an air cleaner, a set-top box, a home automation control panel, a security control panel, a TV box (e.g., Samsung HomeSync™, Apple TV™, or Google TV™), a gaming console (Xbox™, PlayStation™), an electronic dictionary, an electronic key, a camcorder, or an electronic picture frame.

According to other embodiments of the disclosure, examples of the electronic device may include at least one of various medical devices (e.g., diverse portable medical measuring devices (a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning satellite (GPS) receiver, an event data recorder (EDR), a flight data recorder (FDR), an automotive infotainment device, an sailing electronic device (e.g., a sailing navigation device or a gyro compass), avionics, security devices, vehicular head units, industrial or home robots, automatic teller's machines (ATMs), point of sales (POS) devices, or internet of things (IoT) devices (e.g., a bulb, various sensors, an electric or gas meter, a sprinkler, a fire alarm, a thermostat, a street light, a toaster, fitness equipment, a hot water tank, a heater, or a boiler).

According to various embodiments of the disclosure, examples of the electronic device may at least one of part of a piece of furniture or building/structure, an electronic board, an electronic signature receiving device, a projector, or various measurement devices (e.g., devices for measuring water, electricity, gas, or electromagnetic waves). According to various embodiments of the disclosure, the electronic device may be one or a combination of the above-listed devices. According to an embodiment of the disclosure, the electronic device may be a flexible electronic device. According to an embodiment of the disclosure, the electronic device is not limited to the above-listed devices, and may include new electronic devices depending on the development of technology.

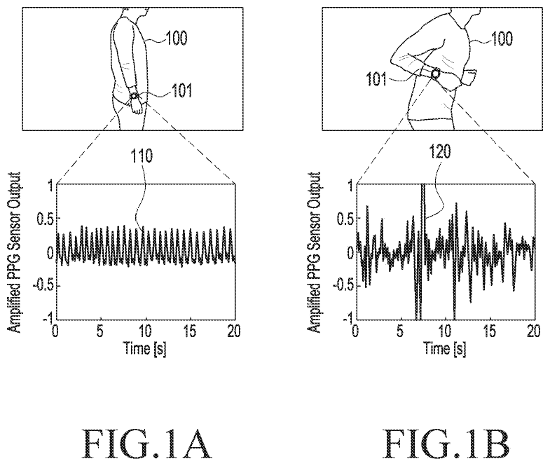

FIGS. 1A and 1B illustrate measuring a physiological signal using a wearable device according to various embodiments of the disclosure. FIG. 1A illustrates a user wearing a wearable device measures a physiological signal in a stable state. FIG. 1B illustrates an example in which the user measures a physiological signal in a moving state.

Referring to FIG. 1A, a wearable device 101 may measure the physiological signal of a user 100 who remains stable without physical motion. A photoplethysmography (PPG) sensor typically used in the wearable device 101 may measure at least one physiological parameter of the heart rate, heart rate variability, stress index, respiration rate, or blood pressure of the user 100 who is in the stable state. The wearable device 101 may provide a physiological signal 110 for the user 100 in the stable condition based on the at least one physiological parameter measured by the PPG sensor. When the user 100 maintains the stable state, the PPG sensor is not influenced by external factors, such as noise, and is thus able to obtain relatively precise physiological signal data. Thus, the physiological signal 110 obtained in the stable state as shown in FIG. 1A may be regarded as reliable data.

Referring to FIG. 1B, the wearable device 101 may measure the physiological signal of the user 100 who is in the moving state where the user makes physical motion (e.g., walking, running, or swimming). The PPG sensor equipped in the wearable device 101 is vulnerable to motion and, upon measuring a physiological signal, the result of measurement may be affected by the posture or motion of the user 100. The physiological signal measured by the PPG sensor while the user 100 maintains the moving state may have motion artifacts. Thus, a physiological signal 120 obtained in the moving state as shown in FIG. 1B may be less reliable than the physiological signal 110 obtained in the stable state.

According to an embodiment of the disclosure, the stable state or the moving state may be differentiated by the signal-to-noise ratio (SNR) or signal quality of the PPG signal measured by the PPG sensor. For example, when the SNR or signal quality of the PPG signal is higher than a designated level, it may be defined as the stable state and, when the SNR or signal quality of the PPG signal is lower than the designated level, it may be defined as the moving state.

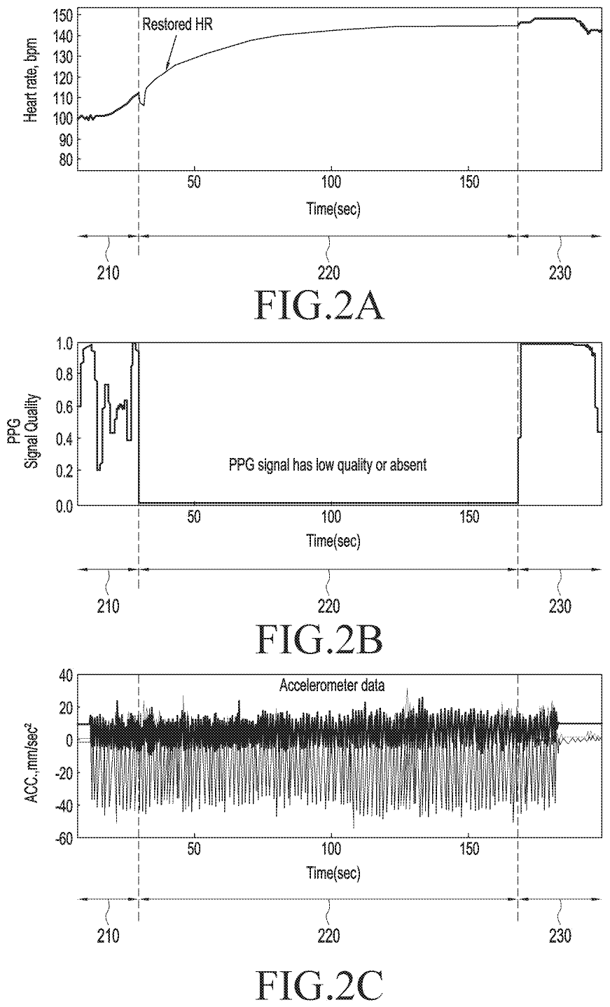

FIGS. 2A, 2B, and 2C illustrate a physiological signal, signal quality and acceleration signal measured corresponding to whether the user moves according to an embodiment of the disclosure.

Referring to FIGS. 2A, 2B, and 2C, the measured physiological signal, signal quality, and acceleration signal show different shapes depending on what state the user wearing the wearable device is in. In FIGS. 2 A, 2B, and 2C, periods 210 and 230 on the time axis may denote time periods for the stable state during which the user makes no physical motion, and period 220 may denote a time period for the moving state during which the user makes physical motion.

In period 210, the wearable device may measure a physiological parameter for the user using the PPG sensor and the degree of the user's motion using an acceleration sensor. Although FIGS. 2A, 2B, and 2C illustrates the results of measurement of heart rate as an example of the physiological parameter, other physiological parameters, such as heart rate variability, stress index, respiration rate, or blood pressure, may be measured according to an embodiment. In period 210, the user is in the stable state but prepares for physical motion and, thus, the acceleration data is recorded as increasing as in FIG. 2C, and the signal quality of PPG signal is irregular as in FIG. 2B. However, although the quality of PPG signal is irregular in period 210, if it remains over a threshold level which is a reference for determination as to whether there is motion, the heart rate recorded in FIG. 2A, as reliable data, may be used for analyzing the user's health condition.

Period 220 may be a time period during which the user makes a predetermined level of, or more, physical motion. The physical motion means that there is such an activity as walking, running, or swimming. In period 220, the amplitude of acceleration data is recorded as varying, corresponding to the user's moving state, as in FIG. 2C, and the signal quality of PPG signal may be recorded as low or the PPG signal may not be recorded as in FIG. 2B. In this case, the heart rate recorded in FIG. 2A may be distorted data that contains motion artifacts and be thus unreliable and unavailable for analysis of the user's health condition. In period 220, the data for heart rate needs to be corrected to precisely reflect the user's moving state.

Period 230 may be a time period during which the user's motion ends and enters back into the stable state. In period 230, the acceleration data may be reduced back to the level prior to the occurrence of motion as in FIG. 2C and, as in FIG. 2B, the signal quality of PPG signal may be recorded as restored to the threshold level or more. In period 230, as the quality of PPG signal recorded is restored to the threshold level or more, the heart rate recorded in FIG. 2A may be used as reliable data.

As such, the steady measurement and analysis of the user's health condition using the wearable device require that physiological signal measurement be performed in different schemes depending on whether the user moves. For example, in the stable state where the SNR or signal quality of PPG signal measured by the PPG sensor in the wearable device is the threshold level or more, the PPG sensor is used to measure the physiological signal and, in the moving state where the SNR or signal quality of PPG signal is less than the threshold level, the physiological signal is estimated based on the motion feature obtained using the acceleration sensor, thereby preventing an error due to motion.

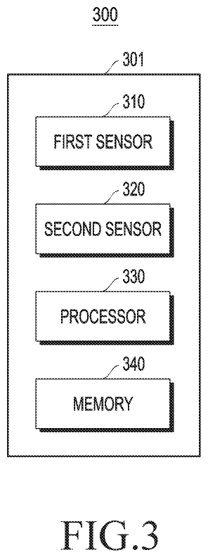

FIG. 3 is a block diagram 300 illustrating an electronic device according to an embodiment of the disclosure.

Referring to FIG. 3, an electronic device 301 may be a portable device (e.g., a wearable device or smartphone) that gathers the user's physiological information or motion information and provides the results of analysis on the gathered information. The electronic device 301 may include a first sensor 310, a second sensor 320, a processor 330, and a memory 340.

Referring to FIG. 3, the first sensor 310 may measure a physiological parameter for the user. The physiological parameter may include at least one of a heart rate, a heart rate variability, a stress index, a respiration rate, or a blood pressure. The first sensor 310 may be a PPG sensor that may measure the physiological parameter according to a designated cycle or continuously.

According to an embodiment of the disclosure, the second sensor 320 may measure the user's motion feature. The motion feature may include at least one of a motion type, a motion intensity, or a motion duration corresponding to the user's motion. The second sensor 320 may include at least one of an accelerometer sensor, a gyroscope sensor, a radar sensor, or a camera.

According to an embodiment of the disclosure, the processor 330 may be operatively connected with the first sensor 310 and the second sensor 320. The memory 340 may be operatively connected with the processor 330.

According to an embodiment of the disclosure, the memory 340 may store instructions to, when executed, enable the processor 330 to perform various operations. For example, the processor 330 may monitor the user's physiological signal based on the physiological parameter measured by the first sensor 310.

The processor 330 may detect the user's motion using at least one of the first sensor 310 or the second sensor 320 while monitoring the user's physiological signal. In response to detecting the user's motion, the processor 330 may identify a first signal indicating a physiological activity trend based on the user's motion feature obtained from the second sensor 320. While the user's motion is detected, the result of measurement of the physiological parameter by the first sensor 310 may have motion artifacts, and thus, the physiological signal may be distorted. Thus, the physiological signal may be measured indirectly using the second sensor 320, which measures the motion feature, instead of the first sensor 310 which is prone to be influenced by the user's posture or operation. For example, while the user's motion is maintained, the processor 330 may identify the user's motion feature using the second sensor 320 and generate the first signal according to the motion feature using a physiological activity prediction model. The physiological activity prediction model may be one for predicting the trend for the physiological parameter indirectly measured considering the motion feature while detecting the user's motion and may be implemented based on machine learning technology. For example, while the user is running, the processor 330 may predict the cardiovascular system according to the running speed and duration obtained via the second sensor 320 using the physiological activity prediction model and may generate a signal regarding the physiological parameter trend based on the result of prediction. The physiological activity prediction model may be downloaded from an external electronic device, stored in the memory 340, or used via real-time communication with the external electronic device.

According to an embodiment of the disclosure, the physiological activity prediction model may predict the user's physiological activity trend based on a machine learning technique including at least one of a support vector machine, a linear regression model, a neural network, or a decision tree. Further, in this process, the physiological activity prediction model may predict the user's physiological activity trend considering personal information including at least one of the user's gender, age, height, weight, athletic ability, or health condition.

The processor 330 may correct the user's physiological signal recorded for the first time period during which the user's motion is maintained based on the first signal. For example, the processor 330 may correct the physiological signal corresponding to the first time period in such a manner as to merge the first signal with the physiological signal measured by the first sensor 310 for the second time period during which the user's motion is not detected. According to an embodiment of the disclosure, the first time period may be a period during which a predetermined level of, or more, user motion is detected, and the second time period may be a period during which the predetermined level of, or more, user motion is not detected. The first time period and the second time period may be differentiated based on the SNR or signal quality of signal measured by the first sensor 310. As another example, the first time period and the second time period may be differentiated based on motion information obtained from the second sensor 320.

According to an embodiment of the disclosure, the electronic device 301 may further include an output means including at least one of a display, speaker, or haptic module for outputting the corrected physiological signal and analysis data therefor and a communication module supporting wireless communication with an external electronic device.

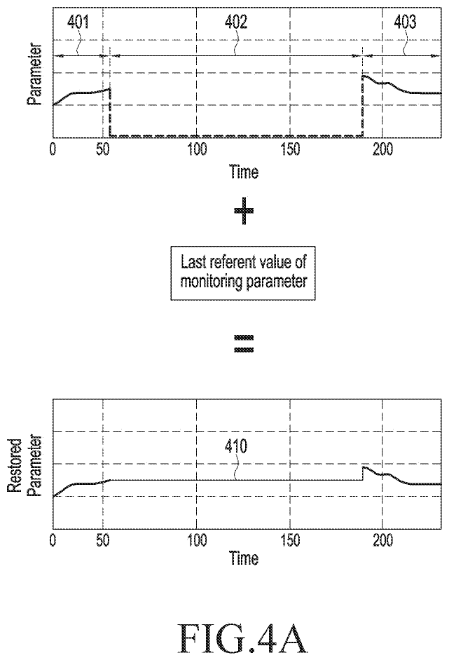

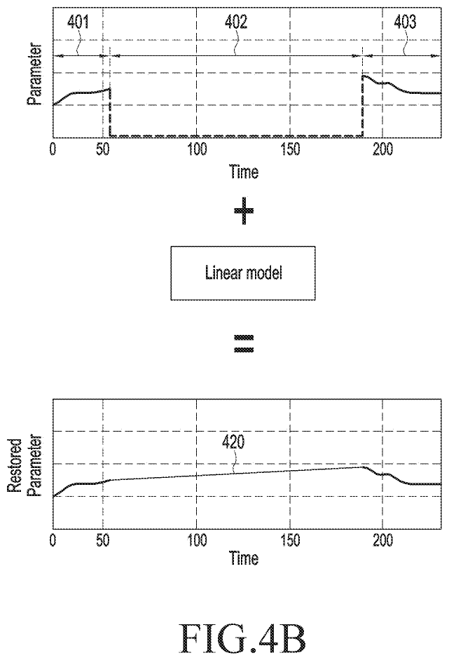

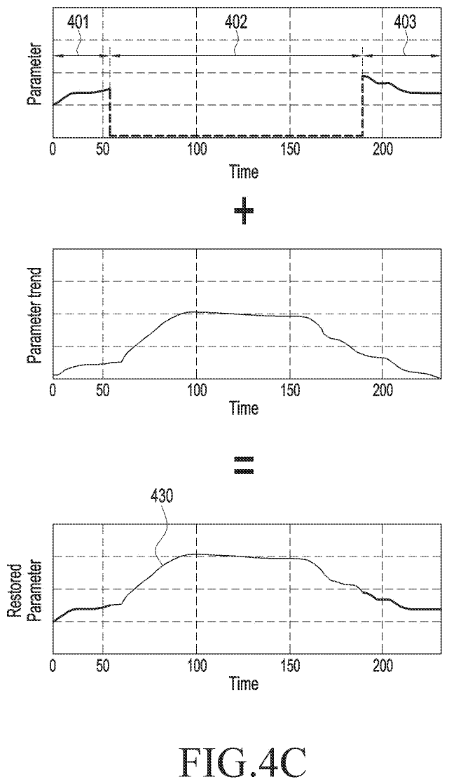

FIGS. 4A, 4B, and 4C illustrate a method of correcting physiological signals when a user moves according to various embodiments of the disclosure.

Referring to FIGS. 4A and 4B illustrate a legacy scheme of correcting the physiological signal measured while the user moves according to the prior art. FIG. 4C illustrates a scheme of correcting a physiological signal considering the motion feature according to an embodiment.

The electronic device 301 may obtain a real-time physiological signal for the user using a first sensor (e.g., a PPG sensor or blood pressure sensor) capable of directly measuring the user's physiological parameter. However, since the first sensor may be influenced by the user's posture or motion, the first sensor may obtain precise measurement results only while the user remains without making a predetermined level of, or more, motion. If the predetermined level of, or more, motion is made, the physiological parameter measured by the first sensor may be distorted by motion artifacts. Thus, the result of measurement in the moving period needs to be corrected to enhance the reliability of physiological signal measurement data and analyzed data.

The graphs at the top of FIGS. 4A to 4C show the physiological signal corresponding to the physiological parameter measured by the first sensor. In the graphs, period 401 on the time axis may denote a time period during which the stable state is maintained before a predetermined level of, or more, motion is detected, period 402 a time period during which the predetermined level of, or more, motion is detected, and period 403 a time period during which the predetermined level of, or more, motion ends and the electronic device enters back into the stable state.

Referring to FIGS. 4A and 4B, the electronic device 301 may correct the physiological signal in the moving period using a reference value for the physiological parameter measured in the stable state. In FIG. 4A, the electronic device 301 may correct the physiological signal in period 402 using the last physiological parameter value measured in period 401, which is before the predetermined level of, or more, motion is detected, as a reference value. In FIG. 4A, the corrected physiological signal 410 for period 402 is shown as maintaining the reference value during period 402. In FIG. 4B, the electronic device 301 may correct the physiological signal for period 402 using a linear model. In this case, the electronic device 301 may obtain the physiological signal 420 for period 402 by using the last value measured in period 401 during which the electronic device is in the stable state before detecting the predetermined level of, or more, motion, as a first reference value, and the first value measured in period 403 during which the predetermined level of, or more, motion ends and the electronic device enters the stable state, as a second reference value, and applying the first reference value and the second reference value to the linear model. In FIG. 4B, the corrected physiological signal 420 for period 402 is shown in the form of linearly connecting the first reference value and the second reference value. In FIGS. 4A and 4B, the corrected physiological signals 410 and 420 for period 402 are determined by a parameter value measured immediately before or after detecting the motion and may thus fail to dynamically reflect the physiological activity trend according to the user's motion feature in period 402.

FIG. 4C illustrates a scheme of correcting a physiological signal in a moving period, with the physiological activity trend per the user's motion feature dynamically reflected.

Referring to FIG. 4C, the electronic device 301 may indirectly measure a physiological signal using a second sensor (e.g., an accelerometer sensor, gyroscope sensor, radar sensor, or camera) measuring the motion feature instead of the first sensor which is influenced by the user's posture or motion. The electronic device 301 may obtain information regarding the motion feature including at least one of the type, intensity, or duration of motion using the second sensor and predict the physiological activity trend based on the obtained motion feature-related information. The predicted physiological activity trend may be shown in the intermediate graph of FIG. 4C. The electronic device 301 may obtain the corrected physiological signal 430 in such a manner as to merge the predicted physiological activity trend for period 402 with period 402 for the physiological signal measured by the first sensor.

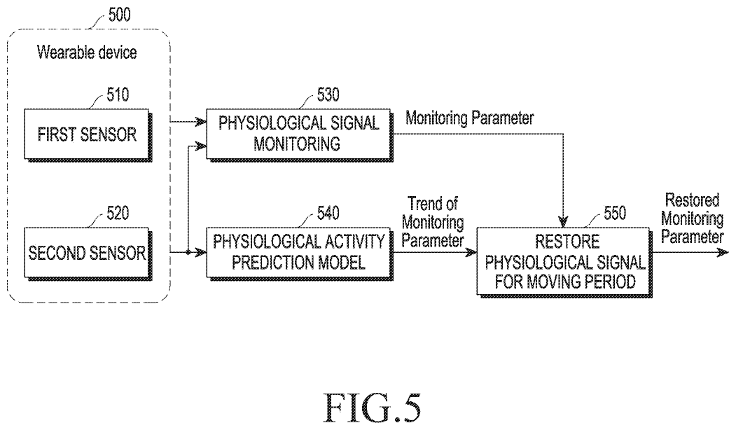

FIG. 5 illustrates a process of estimating a physiological signal in a period during which a user moves according to an embodiment of the disclosure.

Referring to FIG. 5, a wearable device 500 may include a first sensor 510 measuring the user's physiological parameter and a second sensor 520 measuring the user's motion feature. The wearable device 500 of FIG. 5 may correspond to the electronic device 301 of FIG. 3.

The wearable device 500 may obtain at least one parameter for physiological information via physiological signal monitoring 530 using measurement information obtained from the first sensor 510 and the second sensor 520 and generate a physiological signal using the at least one obtained parameter. The first sensor 510 may be a PPG sensor that may measure the user's physiological data according to a designated cycle or continuously. The second sensor 520 may be an acceleration sensor that measures the motion feature, such as the type, intensity, or duration of the user's motion. The first sensor which directly measures the user's physiological data may be influenced by the user's posture or motion and, thus, the first sensor may obtain precise measurement results only while the user remains without making a predetermined level of, or more, motion.

When the predetermined level of, or more, user motion is detected by the first sensor 510 or second sensor 520, the wearable device 500 may generate a physiological signal using the measurement information obtained by the second sensor 520 instead of the measurement information obtained by the first sensor 510 which is unreliable. For example, the wearable device 500 may predict a physiological activity trend in the moving period by applying motion feature-related information obtained by the second sensor 520 to a physiological activity prediction model 540. The physiological activity prediction model 540 may predict the user's physiological activity trend based on a machine learning technique including at least one of a support vector machine, a linear regression model, a neural network, or a decision tree. The wearable device 500 may perform physiological signal restoration 550 while moving in such a manner as to synthesize the predicted physiological activity trend for the moving period with the physiological signal generated as a result of physiological signal monitoring 530 and correct the physiological signal for the moving period according to the physiological signal restoration 550 while moving. For example, the procedure of physiological signal restoration 550 while moving may be performed by an optimization scheme which may be shown in Equation 1 below:

$$ff ( t , shift ) ) = {\int }_{t - 0.5 T}^{t + 0.5 T} [ W ( τ ) SQ ( τ ) {( P \_ est ( τ ) - ( P \_ trend ( τ ) + shift ) )}^{2} ] d τ \\ shif t\_ opt ( t ) = argmin ( ff ( t , shift ) ) \qquad Equation 1$$

Here, W(τ) denotes the weighting window with the maximum value at the current time (t), and T denotes the time period of the weighting window. P_est(τ) and P_trend(τ), respectively, denote the result of physiological parameter monitoring and the physiological parameter trend estimated by the ML model based on data from the second sensor. SQ(τ) is the signal quality vector for reflecting a quality difference between the result of physiological parameter monitoring and the physiological parameter trend. shift denotes the shift between the trend and the physiological parameter monitoring result, and shift_opt(t) denotes the optimal shift for the current time.

According to an embodiment of the disclosure, the wearable device 500 may record the parameter according to the result of physiological signal monitoring 530 as a physiological signal in the time period during which the user maintains the stable state, and the physiological activity trend obtained by the physiological activity prediction model 540 as a physiological signal in the time period during which the user is moving and merge the physiological signals separately recorded in the time periods, thereby generating a physiological signal for the user. Alternatively, the wearable device 500 may synthesize the parameter reference value measured in the user's stable state with the physiological activity trend and reflect the synthesized signal as the physiological signal in the moving state. In this case, the mean of the parameter values measured in the stable state or the measurement value at a designated time (e.g., the last measurement time before motion is detected or the first measurement time after motion ends) may be used as the parameter reference value.

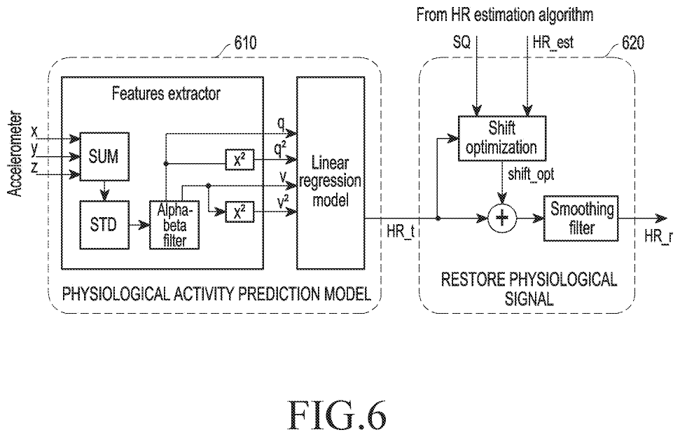

FIG. 6 illustrates a process of estimating a physiological signal using a physiological activity prediction model in a period during which a user moves according to an embodiment of the disclosure.

In the time period during which the user remains in the stable state while remaining and a predetermined level of, or more, motion is not detected, the wearable device 500 may obtain reliable physiological parameter data using a sensor. However, in the moving period during which the predetermined level of, or more, motion is detected, data measured directly using the sensor of the wearable device 500 may be unreliable due to distortion by motion artifacts and thus need to correct in the period.

Referring to FIG. 6, the wearable device 500 may predict the physiological activity trend HR_t in the moving state using a physiological activity prediction model 610. According to an embodiment of the disclosure, the wearable device 500 may summate (SUM) data (x, y, z) obtained from an acceleration sensor, return (STD) a standard deviation, and obtain the predicted physiological activity trend HR_t by an alpha-beta filter and linear regression model. Besides the linear regression model, the physiological activity prediction model 610 may use a machine learning technique, such as a support vector machine, a neural network, or a decision tree so as to predict the physiological activity trend.

The physiological activity trend HR_t predicted by the physiological activity prediction model 610 may be reflected to the physiological signal result by the physiological signal restoration 620. In the block of physiological signal restoration 620, shift optimization of the physiological activity trend HR_t predicted for the moving state period may be performed as in Equation 2 below:

$$ff ( shift ) = {\sum }_{k = 0}^{N - 1} [ {Win}_{k} {SQ}_{k} * {( {HR \_ t}_{est} - ( {HR \_ t}_{k} + shift ) )}^{2} ] \\ Shift \_ opt = argmin ( ff ( shift ) ) \qquad Equation 2$$

Here, HR_est denotes the result of monitoring the data measured by the sensor in the stable state period, and HR_T denotes the physiological activity trend predicted for the moving state period. SQ denotes the signal quality vector for reflecting the quality difference between the monitoring result and the physiological activity trend. Win denotes the Gaussian weighting window with the maximum value, and N denotes the length of the weighting window.

The data shift_opt obtained as a result of the shift optimization may be synthesized with the physiological activity trend HR_T and be then processed by a smoothing filter, so that a restored physiological signal HR_r for the moving period may be obtained. The restored physiological signal HR_r is generated (or is resultant from correction) based on physiological activity trend prediction using the data measured by the acceleration sensor. Thus, the wearable device 500 may provide a physiological signal that dynamically reflects the user's motion feature in the moving period.

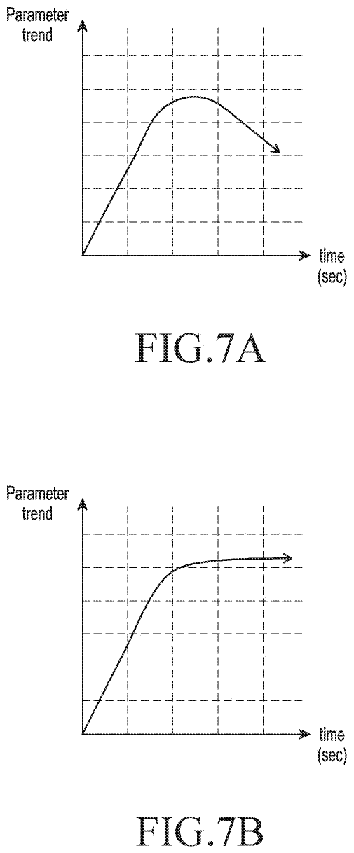

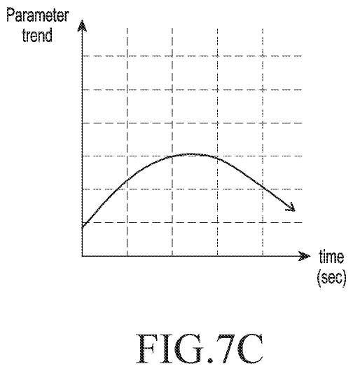

FIGS. 7A, 7B, and 7C illustrate a physiological activity prediction model according to various embodiments of the disclosure.

According to an embodiment of the disclosure, the wearable device 500 may previously generate and store per-motion feature prediction models for predicting a physiological activity trend in the moving period. In this case, upon detecting a predetermined level of, or more, user motion by the first sensor 510 or second sensor 520, the wearable device 500 may select the prediction model corresponding to the motion feature obtained by the second sensor 520 among the pre-stored prediction models and apply information regarding the motion feature obtained by the second sensor 520 to the selected prediction model, thereby predicting the physiological activity trend in the moving period.

Referring to FIGS. 7A, 7B, and 7C, the motion feature may include at least one of the type, intensity, or duration of motion, and the per-motion feature prediction models may be distinguished. For example, when the user's motion feature obtained by the second sensor 520 is identified as short-distance running (e.g., running for one minute), a prediction model as shown in FIG. 7A may be applied which draws a sharp-rise-sharp-fall curve. When the user's motion feature obtained by the second sensor 520 is identified as long-distance running (e.g., running for 30 minutes), a prediction model as shown in FIG. 7B may be applied which draws a sharp-rise-then-flat curve. When the user's motion feature obtained by the second sensor 520 is identified as one-minute walking or cycling which is of relatively low intensity, a prediction model as shown in FIG. 7C may be applied which draws a gentle-rise-gentle-fall curve.

Use of the pre-stored prediction models as shown in FIGS. 7A to 7C may reduce the complexity of the physiological activity trend prediction per the user's motion and shorten the time required for physiological signal correction and physiological activity trend prediction for the moving period.

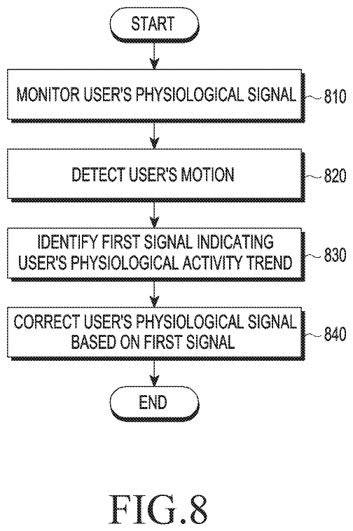

FIG. 8 is a flowchart illustrating a method of estimating a physiological signal by an electronic device according to an embodiment of the disclosure. According to an embodiment of the disclosure, the electronic device 301, as a portable device (e.g., a wearable device or smartphone), may gather the user's physiological information or motion information and provide a result of analysis of the gathered information. To that end, the electronic device 301 may include a first sensor 310 measuring the user's physiological parameter and a second sensor 320 measuring the user's motion feature.

Referring to FIG. 8, at operation 810, the electronic device 301 may monitor the user's physiological signal based on the physiological parameter measured by the first sensor 310. The first sensor 310 may be a PPG sensor that may measure the physiological parameter according to a designated cycle or continuously. The physiological parameter may include at least one of a heart rate, a heart rate variability, a stress index, a respiration rate, or a blood pressure.

At operation 820, the electronic device 301 may detect the user's motion using at least one of the first sensor 310 or the second sensor 320 while monitoring the user's physiological signal. The second sensor 320 may include at least one of an accelerometer sensor, a gyroscope sensor, a radar sensor, or a camera. The motion feature may include at least one of a motion type, a motion intensity, or a motion duration corresponding to the user's motion.

At operation 830, in response to detecting the user's motion of operation 720, the electronic device 301 may identify a first signal indicating a physiological activity trend based on the user's motion feature obtained from the second sensor 320. While the user's motion is detected, the result of measurement of the physiological parameter by the first sensor 310 may have motion artifacts, and thus, the physiological signal may be distorted. Thus, the physiological signal may be measured indirectly using the second sensor 320, which measures the motion feature, instead of the first sensor 310 which is prone to be influenced by the user's posture or operation. At operation 830, the electronic device 301 may identify the user's motion feature using the second sensor 320 and generate the first signal according to the motion feature using a physiological activity prediction model. The physiological activity prediction model may be one for predicting the trend for the physiological parameter indirectly measured considering the motion feature while detecting the user's motion and may be implemented based on machine learning technology including at least one of a support vector machine, a linear regression model, a neural network, or decision tree. The physiological activity prediction model may predict the user's physiological activity trend considering personal information including at least one of the user's gender, age, height, weight, athletic ability, or health condition. At operation 830, the electronic device 301 may use the physiological activity prediction model previously stored in the memory 330 or via communication with an external electronic device.

At operation 840, the electronic device 301 may correct the user's physiological signal recorded for the first time period during which the user's motion is maintained based on the first signal. At operation 840, the electronic device 301 may correct the physiological signal corresponding to the first time period by merging the first signal with the physiological signal measured by the first sensor 310 for the second time period during which the user's motion is not detected. The first time period, as a period during which a predetermined level of, or more, user motion is detected, may mean that the SNR or signal quality of signal measured by the first sensor 310 is a designated level or more or motion information obtained by the second sensor 320 denotes the presence of the predetermined level of, or more, user motion. The second time period, as a period during which the predetermined level of, or more, user motion is not detected, may mean that the SNR or signal quality of signal measured by the first sensor 310 is less than the designated level or motion information obtained by the second sensor 320 denotes the absence of the predetermined level of, or more, user motion.

After operation 840, the electronic device 301 may output the corrected physiological signal and analysis data therefor, using at least one output means of a display, a speaker, or a haptic module.

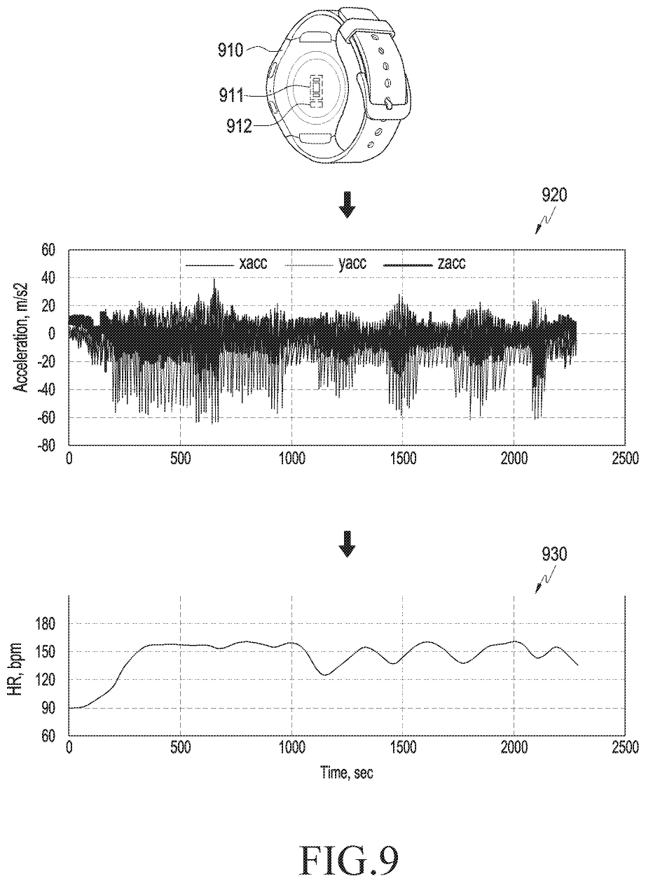

FIG. 9 illustrates providing physiological information estimated corresponding to a motion feature by a smartwatch according to an embodiment of the disclosure.

Referring to FIG. 9, a smartwatch 910 is a device that is worn on the user's body part to gather information about the user and provides a result of analysis of the gathered information. The smartwatch 910 may correspond to the electronic device 301 of FIG. 3 or the wearable device 500 of FIG. 5.

The smartwatch 910 may include a first sensor 911 and a second sensor 912. According to an embodiment of the disclosure, the first sensor 911 may be a PPG sensor that measures the user's physiological parameter according to a predetermined cycle. The second sensor 912 may be an acceleration sensor that measures the user's motion feature.

In the stable state in which the user wearing the smartwatch 910 remains without making a predetermined level of, or more, motion, the smartwatch 910 may obtain a physiological signal based on at least one physiological parameter of the heart rate, heart rate variability, stress index, respiration rate, or blood pressure measured using the signal from the first sensor 911.

In the moving state in which the signal from the first sensor 911 may be distorted by motion artifacts, the smartwatch 910 may predict the physiological activity trend for the moving period based on the motion feature obtained by the second sensor 912 and estimate a physiological signal based on the predicted trend. In FIG. 9, graph 920 denotes the acceleration data measured using the second sensor in the moving period, and graph 930 denotes the physiological signal data in the moving period, based on the measured acceleration data, with the predicted physiological activity trend reflected. The acceleration data of graph 920 may be measured as differing depending on the motion feature, such as the type, intensity, or duration of the user's motion and, thus, the physiological activity trend in the moving state may be varied as well. Thus, it may be identified that the result of physiological signal estimation shown in graph 930 is also dynamically varied.

The electronic device according to various embodiments may be one of various types of electronic devices. The electronic devices may include, for example, a portable communication device, a computer device, a portable multimedia device, a portable medical device, a camera, a wearable device, or a home appliance. According to an embodiment of the disclosure, the electronic device is not limited to the above-listed embodiments.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment of the disclosure, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the wearable device 101). For example, a processor of the machine (e.g., the wearable device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment of the disclosure, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments of the disclosure, each component (e.g., a module or a program) of the above-described components may include a single entity or multiple entities. According to various embodiments of the disclosure, one or more of the above-described components may be omitted, or one or more other components may be added. Alternatively or additionally, a plurality of components (e.g., modules or programs) may be integrated into a single component. In such a case, according to various embodiments of the disclosure, the integrated component may perform one or more functions of each of the plurality of components in the same or similar manner as they are performed by a corresponding one of the plurality of components before the integration. According to various embodiments of the disclosure, operations performed by the module, the program, or another component may be carried out sequentially, in parallel, repeatedly, or heuristically, or one or more of the operations may be executed in a different order or omitted, or one or more other operations may be added.

According to an embodiment of the disclosure, there is provided a storage medium storing instructions executed to enable at least one processor to perform at least one operation that may include a method of estimating a physiological signal by an electronic device including a first sensor configured to measure a user's physiological parameter and a second sensor configured to measure the user's motion feature, the method comprising monitoring (810) the user's physiological signal based on the physiological parameter measured by the first sensor, detecting (820) the user's motion using at least one of the first sensor or the second sensor while monitoring the user's physiological signal, identifying (830) a first signal indicating a physiological activity trend based on the user's motion feature obtained from the second sensor in response to detecting the user's motion, and correcting (840) the user's physiological signal recorded for a first time period during which the user's motion is maintained based on the first signal.

As is apparent from the foregoing description, according to various embodiments of the disclosure, the electronic device and method may estimate physiological signals considering the physiological activity trend according to the motion feature, instead of the PPG signal, when motion artifacts occur due to the user's motion, thereby raising the accuracy and reliability for physiological information obtained using the wearable device. Further, the electronic device and method enable low-power monitoring of physiological signals by using an accelerometer sensor, instead of a PPG sensor.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a first sensor configured to measure a physiological parameter of a user;
a second sensor configured to measure motion feature of the user;
at least one processor operatively connected with the first sensor or the second sensor; and
a memory operatively connected with the at least one processor, wherein the memory stores instructions executed to enable the at least one processor to:
monitor a physiological signal of the user based on the physiological parameter measured by the first sensor, upon detecting motion of the user using at least one of the first sensor or the second sensor while monitoring the physiological signal,
identify a first signal indicating a physiological activity trend based on the motion feature obtained from the second sensor, and
correct the physiological signal recorded for a first time period during which the motion is maintained, based on the first signal.

2. The electronic device of claim 1, wherein the first sensor includes a photoplethysmogram (PPG) sensor configured to measure the physiological parameter continuously or according to a designated cycle.

3. The electronic device of claim 2, wherein the physiological parameter includes at least one of a heart rate, a heart rate variability, a stress index, a respiration rate, or a blood pressure.

4. The electronic device of claim 1, wherein the second sensor includes at least one of an accelerometer sensor, a gyroscope sensor, a radar sensor, or a camera.

5. The electronic device of claim 1, wherein the instructions enable the at least one processor to identify the motion feature using a second sensor for the first time period and generate the first signal according to the motion feature using a physiological activity prediction model stored in the memory.

6. The electronic device of claim 5, wherein the motion feature includes at least one of a motion type, a motion intensity, or a motion duration corresponding to the motion.

7. The electronic device of claim 5, wherein the physiological activity prediction model is configured to predict the physiological activity trend based on a machine learning technique including at least one of a support vector machine, a linear regression model, a neural network, or a decision tree.

8. The electronic device of claim 5, wherein the physiological activity prediction model is configured to predict the physiological activity trend considering personal information including at least one of the user's gender, age, height, weight, athletic ability, or health condition.

9. The electronic device of claim 1, wherein the instructions enable the at least one processor to merge the physiological signal recorded for a second time period during which the motion is not detected with the first signal to thereby correct the physiological signal corresponding to the first time period.

10. The electronic device of claim 1, further comprising a display, wherein the instructions enable the at least one processor to output the corrected physiological signal on the display.

11. A method of estimating a physiological signal by an electronic device including a first sensor configured to measure a physiological parameter of a user and a second sensor configured to measure motion features of the user, the method comprising:
monitoring a physiological signal of the user based on the physiological parameter measured by the first sensor;
detecting motion of the user using at least one of the first sensor or the second sensor while monitoring the physiological signal;
identifying a first signal indicating a physiological activity trend based on the motion feature obtained from the second sensor in response to detecting the motion of the user; and
correcting the physiological signal recorded for a first time period during which the motion is maintained, based on the first signal.

12. The method of claim 11, wherein the first sensor includes a photoplethysmogram (PPG) sensor configured to measure the physiological parameter continuously or according to a designated cycle.

13. The method of claim 12, wherein the physiological parameter includes at least one of a heart rate, a heart rate variability, a stress index, a respiration rate, or a blood pressure.

14. The method of claim 11, wherein the second sensor includes at least one of an accelerometer sensor, a gyroscope sensor, a radar sensor, or a camera.

15. The method of claim 11, wherein the identifying of the first signal includes identifying the motion feature using a second sensor for the first time period and generating the first signal according to the motion feature using a physiological activity prediction model stored in a memory of the electronic device.

16. The method of claim 15, wherein the motion feature includes at least one of a motion type, a motion intensity, or a motion duration corresponding to the motion.

17. The method of claim 15, wherein the generating of the first signal according to the motion feature using the physiological activity prediction model includes predicting the physiological activity trend based on a machine learning technique including at least one of a support vector machine, a linear regression model, a neural network, or a decision tree.

18. The method of claim 15, wherein the generating of the first signal according to the motion feature using the physiological activity prediction model includes predicting the physiological activity trend considering personal information including at least one of the user's gender, age, height, weight, athletic ability, or health condition.

19. The method of claim 11, wherein the correcting of the physiological signal includes merging the physiological signal recorded for a second time period during which the motion is not detected with the first signal to thereby correct the physiological signal corresponding to the first time period.

20. The method of claim 11, further comprising outputting the corrected physiological signal on a display.
